(19) United States
(12) Patent Application Publication
(10) Pub. No.: US 2022/0249334 A1
(43) Pub. Date: Aug. 11, 2022

(54) Moisturizer Sheets

(71) Applicants: Bernice Venable, Waldorf, MD (US); Nico Adams, White Plains, MD (US)

(72) Inventors: Bernice Venable, Waldorf, MD (US); Nico Adams, White Plains, MD (US)

(21) Appl. No.: 17/321,697

(22) Filed: May 17, 2021

Related U.S. Application Data
(60) Provisional application No. 63/147,418, filed on Feb. 9, 2021.

(51) Int. Cl. A61K 8/02; A61K 8/92

(52) U.S. Cl. CPC A61K 8/0208; A61K 2800/592; A61K 2800/87; A61K 8/922

(57) ABSTRACT

This present invention relates to a pre-packaged set of oil infused moisturizer sheets that can be used to transfer the oils to a selected area of the body. The moisturizer sheets are infused with different types of oils such as castor oil, almond oil, essential oils, coconut oil, and more. The sheets can be applied multiple times a day to help moisturize user's skin and can be stored in a protective storage case to keep the sheets fresh while offering a simple and convenient method for transporting the sheets. The protective storage case is reusable and can be refilled with a replenishment set of infused moisturizer sheets.

### CROSS-REFERENCE TO RELATED APPLICATION

The present application claims priority to, and the benefit of, U.S. Provisional Application No. 63/147,418, which was filed on Feb. 9, 2021 and is incorporated herein by reference in its entirety.

### FIELD OF THE INVENTION

The present invention relates generally to the field of moisturizing wipes or sheets. More specifically, the present invention relates to a moisturizer sheet infused with various oils such as mineral oil, castor oil, almond oil, essential oils, coconut oil, and more. The oil-infused moisturizer sheets can be applied to any area of the body to moisturize the skin. Additionally, the moisturizer sheets can be stored in a protective storage case to keep the sheets fresh, prohibiting evaporation, and offering a simple and convenient method for transporting the sheets or wipes. Accordingly, this disclosure makes specific reference thereto the present invention. Nonetheless, it is to be appreciated that aspects of the present invention are also equally applicable to other like applications, devices and methods of manufacture.

### BACKGROUND OF THE INVENTION

By way of background, individuals may use different kinds of oils to moisturize and hydrate their skin. Moisturizing the body and face are a part of a daily routine for many individuals. Individuals with particularly dry skin may desire to use oils on their body to keep their skin moisturized. Further, during the wintertime, individuals with normal skin type may suffer from dry skin issues, and may opt for treatments of oils and moisturizers to overcome dryness.

Usually, various oils and moisturizers are available in the market for preventing dryness during wintertime and to moisturize dry skin year round. These oils are packaged and stored in various containers such as bottles, jars and tubes. Individuals typically keep these oil bottles on a countertop, sink area, etc., and may use the oil to moisturize their body as required. During use of the oil, individuals may open the cap of the bottle, dispense the required amount of oil, and then apply the dispensed oil over different areas of the body. However, some individuals may forget to close the cap of the bottle, thereby leaving the oil bottle in an open condition. In such a scenario, the bottles are prone to being knocked over and this can lead to spilling of the oil stored in the bottle, thereby creating a mess and wastage. The mess requires cleaning and the wastage requires replenishment.

Additionally, with conventional bottles, individuals may inadvertently dispense excessive amounts of oil, which leads to wastage of the oil contained in the bottle. This also causes overconsumption and excessive use of the oils, which causes the user to have to frequently replenish expensive oils.

Further, different types of moisturizing oils have different advantages for the user. Individuals may buy multiple oils packaged in separate bottles to gain various advantages from each oil. Buying multiple oils may get expensive for the users. Furthermore, finding storage space for multiple bottles of moisturizing oils can be frustrating. Also, handling multiple oil bottles may be inconvenient for the users.

Therefore, there exists a long felt need in the art for a moisturizing product which moisturizes the body of the user. There is also a long felt need in the art for a moisturizing product which uses a combination of different kinds of oils, and provides advantages of all the constituent oils. Additionally, there is a long felt need in the art for a moisturizing product which can be easily stored and kept at home. Moreover, there is a long felt need in the art for a moisturizing product which can be stored in portable containers and can be conveniently used. Further, there is a long felt need in the art for a moisturizing product packaged in a manner that prevents inadvertent or accidental spilling of oils from the product package or container. Furthermore, there is a long felt need in the art for a moisturizing product which allows the user to dispense appropriate amounts of oil for moisturizing and hydrating the skin, while preventing excessive use of the oils and wastage of the product. Also, there is a long felt need in the art for a moisturizing product which can be conveniently applied to the user's body. Finally, there is a long felt need in the art for a cost-effective moisturizing product which does not require the users to buy multiple oil bottles and enables the users to easily handle the moisturizing products.

The subject matter disclosed and claimed herein, in one potential embodiment, comprises an oil-infused body moisturizer sheet designed to apply infused oil on various body parts of a user. The infused sheets comprise: fibrous, paper, cloth sheet, woven or non-woven material infused with at least one of mineral oil, castor oil, almond oil, and coconut oil in a combination or composition to moisturize the skin. Each sheet provides an effect similar to standard oil from a bottle. Further, the sheet is stored in a protective storage case to keep the sheet fresh and wet while offering a simple and convenient method for transporting the sheet.

In this manner, the novel oil-infused body moisturizer sheet of the present invention accomplishes all of the forgoing objectives, and provides a relatively easy, convenient and cost-effective solution for moisturizing and hydrating a user's skin. The oil-infused body moisturizer sheet of the present invention features different kinds of oils and provides advantages of all the constituting ingredients. The oil-infused body moisturizer sheet is also user friendly, inasmuch as it is less expensive than other alternatives, and does not require the user to expend a great deal of effort or labor into applying the product over the user's body. Additionally, the oil-infused body moisturizer sheets can be conveniently stored in a protective storage case, which enables the moisturizer sheets to be transported easily and prevents wastage of the product.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one potential embodiment, comprises an oil-infused body moisturizer sheet designed to apply infused oil on various body parts of a user. The moisturizer sheet comprises a fibrous, paper, cloth sheet, woven or non-woven material sheet; and, the sheet is infused with at least one of mineral oil, castor oil, almond oil, and coconut oil in a combination to moisturize the skin. Each sheet provides an effect similar to standard oil from a bottle. Further, the sheet can be stored in a protective storage case to keep the sheet fresh and protected from ambient air, while offering a simple and convenient method for transporting a multitude of sheets.

In a further embodiment of the present invention, an oil-infused body moisturizer sheet designed to apply infused oil on the various body parts of a user is disclosed and comprises: a fibrous, paper, cloth sheet, woven or non-woven material sheet; the sheet infused with at least one of mineral oil, castor oil, almond oil, coconut oil, lemon oil, lavender oil, rosemary oil, and tea tree oil; and, the sheet further infused with a fragrance and aqua.

In yet another embodiment of the present invention, a method of applying oil on any part of the body using a pre-infused oil body moisturizer sheet of the present invention is disclosed. The method comprising: selecting a moisturizer sheet infused with a specific oil or a combination of oils as per the requirements of the user; opening a sealed lid of a container or dispenser and pulling out an individual sheet through an opening of the container; holding the sheet with one or both hands and wiping the sheet onto the desired body part such that the oil infused in the sheet is applied to the body part; and, disposing of the used sheet by throwing it in a dustbin or flushing it down the toilet.

The oil-infused body moisturizer sheet is made up of biodegradable and flushable material, and is eco-friendly. The oil-infused body moisturizer sheet is dispensed through a sealed and resealable container which protects the sheets from evaporation, and thereby prevents the sheets from drying out. The resealable packing container helps to retain the oil and moisture of the oil-infused body moisturizer sheets for a long period of time.

The oil moisturizer sheets of the present invention are particularly advantageous as they can be used and applied to any part of the body conveniently and are effective in applying oil in order to moisturize and nourish the skin surface in a single application. The sheets of the present invention replace standard moisturizer oil containers which can be at risk for spills on items like clothing, floors, carpet, furniture, etc. Each sheet application effectively provides similar benefits of standard oil applications from a bottle, while preventing any chance of spills and accompanying mess. The invention further allows users to choose from multiple different sheets infused with various oils like baby oil, castor oil, almond oil, essential oils, coconut oil, and more.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of essential oils infused body moisturizer sheets of the present invention in accordance with the disclosed structure;

FIG. 2 illustrates a diagrammatic representation showing the oil types used for infusing the body moisturizer sheet of the present invention;

FIG. 3 illustrates a diagrammatic representation showing the oil types used for infusing the body moisturizer sheet as per another embodiment of the present invention;

FIG. 4 illustrates a perspective view of another embodiment of an oil-infused body moisturizer sheet of the present invention in accordance with the disclosed structure;

FIG. 5 illustrates another embodiment of oil-infused body moisturizer sheet of the present invention in accordance with the disclosed structure;

FIG. 6 illustrates oil-infused body moisturizer sheet of the present invention used for applying oil on legs of a user;

FIG. 7 illustrates oil-infused body moisturizer sheet of the present invention used for applying oil on arms of a user; and

FIG. 8 illustrates oil-infused body moisturizer sheet of the present invention stored in protective storage case to keep the sheets fresh and accessed from an opening of the protective storage case.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a moisturizing product which moisturizes the body of the user. There is also a long felt need in the art for a moisturizing product which uses a combination of different kinds of oils and provides advantages of all the constituent oils. Additionally, there is a long felt need in the art for a moisturizing product which can be easily stored and kept at home. Moreover, there is a long felt need in the art for a moisturizing product which can be stored in portable containers, which can be conveniently used. Further, there is a long felt need in the art for a moisturizing product packaged in a manner that prevents inadvertent spilling of oils from the product package or container. Furthermore, there is a long felt need in the art for a moisturizing product which allows the user to use the appropriate amount of oil for moisturizing and hydrating the skin, while preventing excessive use of the oils and wastage of the product. Also, there is a long felt need in the art for a moisturizing product which can be conveniently applied to the user's body. Finally, there is a long felt need in the art for a cost-effective moisturizing product which does not require the users to buy multiple oil bottles and enables the users to easily handle the moisturizing products.

The present invention, in one exemplary embodiment, is a novel oil-infused moisturizer sheet designed to apply infused oil on body parts of a user. The infused moisturizer sheets comprise: fibrous, paper, cloth sheet, woven or non-woven material sheet; and, the sheet is infused with at least one of mineral oil (e.g., baby oil), castor oil, almond oil and coconut oil in a combination composition to moisturize the skin. Each sheet provides an effect similar to standard oil from a bottle. Further, the sheet is stored in a protective storage case to keep the sheet fresh, while offering a simple and convenient method for transporting the sheet.

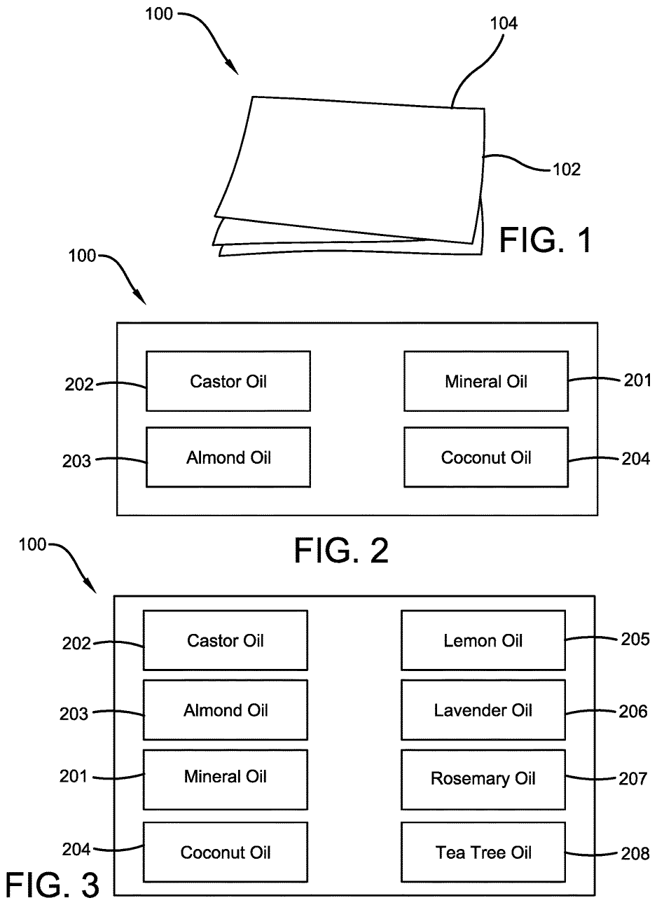

Referring initially to the drawings, FIG. 1 illustrates a perspective view of a carrier and essential oils infused body moisturizer sheet of the present invention in accordance with the disclosed structure. As shown in the figure, each oil-infused body moisturizer sheet is generally rectangular or square in shape, and may have an approximate desired length 102 and width 104. As an example, the moisturizer sheet 100 of standard size may be of 180 mm in length and 150 mm in width. The size of the moisturizer sheet 100 is not restricted and may be of other sizes such as 100 mm to about 350 mm in length, and 70 mm to about 250 mm in width. The dimensions of the moisturizer sheet 100 are not limited and the moisturizer sheet 100 can be manufactured in various shapes and sizes as desired and preferred by the users.

The oil-infused body moisturizer sheet 100 may be manufactured using materials comprising cellulosic fibers, thermoplastic fibers, fibrous material, paper, cloth sheets, woven or non-woven materials, or others. The oil-infused body moisturizer sheet 100 of the present invention resembles and functions similar to a conventional wet wipe. The moisturizer sheets 100 may be packed as a stack of individual wipes placed one over the other or may include a continuous strip of material which has perforations between each sheet 100 and may be rolled into a roll for dispensing.

FIG. 2 illustrates a diagrammatic representation showing, in one potential embodiment, oil types used for infusing the body moisturizer sheet of the present invention. Each oil-infused body moisturizer sheet 100 may be infused with at least one of mineral or baby oil 201, castor oil 202, almond oil 203 and coconut oil 204. The body moisturizer sheet 100 replaces standard moisturizer oil containers and can be used to moisturize skin and hair on any area of the body. Each sheet 100 can be used to wipe any area of the body, thereby effectively transferring the infused oils thereto.

Infusing oils to be used in the present invention are described in detail below. In one exemplary arrangement, a combination of carrier oils and essential oils can be used to infuse the sheets. A first carrier oil comprises mineral oil. Mineral oil is effective at moisturizing the skin and includes rejuvenating and restorative properties to heal dry skin issues like cracked heels. Mineral oil treats and prevents dry, rough, scaly, itchy skin and other minor skin irritations. Mineral oil is an inert substance, which means it doesn't change or react when it comes into contact with other substances. When applied, mineral oil softens and soothes skin. Mineral oil is typically not a suitable carrier oil for essential oils when stored in a liquid formula. Infusing sheets with mineral oil, other carrier oils, and essential oils overcomes the less than desirable characteristics of liquid stored mineral oil and essential oils.

Other carrier oils exist that are superior to mineral oil in the nutritive and emollient benefits that they offer. Within the scope of holistic aromatherapy, carrier oils are compatible with the underlying intent to use pure, botanical ingredients. One such carrier is castor oil. Castor oil used for infusing the sheet 100 is extracted from the seeds of the Ricinus communis plant. The seeds contain a toxic enzyme called ricin. However, the heating process that castor oil undergoes deactivates it, allowing the oil to be used safely. Castor oil has a number of medicinal, industrial and pharmaceutical uses. Castor oil has natural antiviral and antimicrobial properties, and is used for moisturizing and providing skin treatment. Castor oil increases absorption in the skin and is used in the treatment of various skin conditions, including dermatosis, psoriasis, and acne. Castor oil can also be used on its own as a natural alternative to store-bought moisturizers and lotions. Further, castor oil is inexpensive and can be used on the face and body. Castor oil is thick, thus, the present invention provides for the addition of other carrier oils and essential oils to form an ultra-hydrating moisturizer sheet. Mixing castor oil with other oils makes it easier for infusing.

Another carrier oil is almond oil. Almond oil moisturizes and pampers the skin. Almond oil doesn't stick to the skin and does not leave any residue. Almond oil heals minor cuts and injuries while soothing the skin. Almond oil acting as a carrier oil, has the ability to carry other essential oils more deeply into the skin. To be described in more detail hereinafter, mixing almond oil with an essential oil provides additional benefits.

Still yet another carrier oil is coconut oil. Cold-pressed coconut oil is used to infuse the body sheets 100 and can cleanse, hydrate, and soothe the skin. The body sheet infused with coconut oil can be used as rash relief or make-up remover and can be used for both adults and children. Coconut oil is a plant-derived, natural oil. Coconut oil is a vegetable-based carrier oil that has been derived from the seeds, kernels, or nuts of a plant. Coconut oil is a fantastic carrier oil to use when diluting essential oils for topical use. In potential embodiments to be described herein below, each body sheet 100 may be infused with a single oil or a combination of one or more oils to increase the effectiveness and advantages inherent thereto. It should be appreciated that fragrance oils and aqua can also be added to infuse in each body sheet 100.

FIG. 3 illustrates a diagrammatic representation showing the oil types used for infusing the body moisturizer sheet as per another embodiment of the present invention. As represented in FIG. 3, any combination of two or more of mineral oil 201, castor oil 202, almond oil 203, coconut oil 204, lemon oil 205, lavender oil 206, rosemary oil 207, and tea tree oil 208 is infused in the body sheet 100. Along with the oils, aqua, essential oils, natural fragrances may be added to the infused composition.

FIG. 3 represents the introduction of using essential oils in the infusion process. Essential oils are distilled from the aromatic leaves, bark and roots of plants. An essential oil is a concentrated hydrophobic liquid containing volatile (i.e., easily evaporated at normal temperatures) chemical compounds from plants. Essential oils are also known as volatile oils, ethereal oils, aetheroleum, or simply as the oil of the plant from which they were extracted, such as oil of clove. An essential oil is “essential” in the sense that it contains the “essence of” the plant's fragrance—the characteristic fragrance of the plant from which it is derived. Essential oils are generally extracted by distillation, often by using steam. Essential oils should not be confused with perfume, fragrance, etc. as the latter usually include pure chemical components whereas essential oils are derived from plants. Improper use of essential oils may cause harm including allergic reactions, inflammation and skin irritation, and children may be particularly susceptible to the toxic effects of improper use. Essential oils are usually lipophilic (literally: “oil-loving”) compounds that are immiscible (not miscible) with water. The most common way to safely dilute essential oils for topical use is in one or more carrier oils. In exemplary embodiments, the essential oil content is generally from about 0.5% to about 10%. The most popular carrier oils for skin care are jojoba, coconut, wheat germ, olive and avocado. Most common essential oils such as lavender, peppermint, tea tree oil, patchouli and eucalyptus are distilled. Raw plant material, consisting of the flowers, leaves, wood, bark, roots, seeds or peel, is put into an alembic (distillation apparatus) over water. As the water is heated, the steam passes through the plant material, vaporizing the volatile compounds. The vapors flow through a coil, where they condense back to liquid, which is then collected in the receiving vessel.

As described above, carrier oils are used to dilute essential oils and absolutes before they are applied to the skin. They are named carrier oils because they carry the essential oil onto the skin. Diluting essential oils is a critical safety practice when using essential oils. Essential oils alone are volatile; they begin to dissipate as soon as they are applied. The rate of dispersion will vary based on the weight of the carrier oil. Carrier oils do not contain a concentrated aroma, unlike essential oils. Carrier oils do not evaporate like essential oils, which are more volatile. The carrier oils used should be as natural and unadulterated as possible. Many individuals feel organic oils are of higher quality. Cold-pressing and maceration are the two main methods of producing carrier oils. FIG. 3 displays several essential oils to be described hereinbelow. Lemon oil 205 improves skin quality, provides vitamin C, relieves pain, treats acne, heals wounds and lifts the spirit and mood of the user. Further, lemon oil balances the skin's natural pH. Lavender oil 206 has antiseptic and antimicrobial properties, promotes hair growth, and heals minor burns and bug bites. Further, lavender oil 206 helps ease muscles, joint pain, headache, sprains and backache. Rosemary oil 207 eases muscle stress, increases blood circulation, reduces joint inflammation, stimulate follicles and strengthens hair from the root. Tea tree oil 208 acts as a natural deodorant, fights acne, is used as an antiseptic and is also an insect repellant. Tea tree oil 208 possesses antibacterial, anti-inflammatory, antiviral and antifungal properties. If undiluted, tea tree oil can be irritating, so diluting it in another oil like mineral oil or sunflower oil is the best way to use it. Tea tree oil can help soothe dry skin by reducing itching and irritation. A few drops of tea tree oil mixed into a carrier oil provides for an effective moisturizer. One exemplary moisturizer mixture is castor oil and tea tree oil. Both oils have their benefits, which are enhanced after combining them. Referring to the table below, there is disclosed a moisturizer composition that can be formulated with a number of different components in varying quantities in order to achieve a desired result. The moisturizer composition, in one potential embodiment, is provided as a liquid for infusing sheets. Exemplary embodiments for the infusing liquid can be developed using the ingredients and percentages listed below.

| Component | Content in percentage by weight (% w/w) |
|---|---|
| Mineral oil | 10-99 |
| Castor oil | 10-99 |
| Almond oil | 20-80 |
| Coconut oil | 20-80 |
| Lemon oil | 0.5-10.0 |
| Lavender oil | 0.5-10.0 |
| Rosemary oil | 0.5-10.0 |
| Tea Tree oil | 0.5-10.0 |

Infusing the sheets as described herein can be as simple as mixing the carrier oils and essential oils together in a suitable container, placing the sheets into the container, soaking the sheets in the oils until fully saturated, removing the sheets and allowing the excess oils to drain therefrom, and packaging the sheets in any variety of packages as described hereinafter.

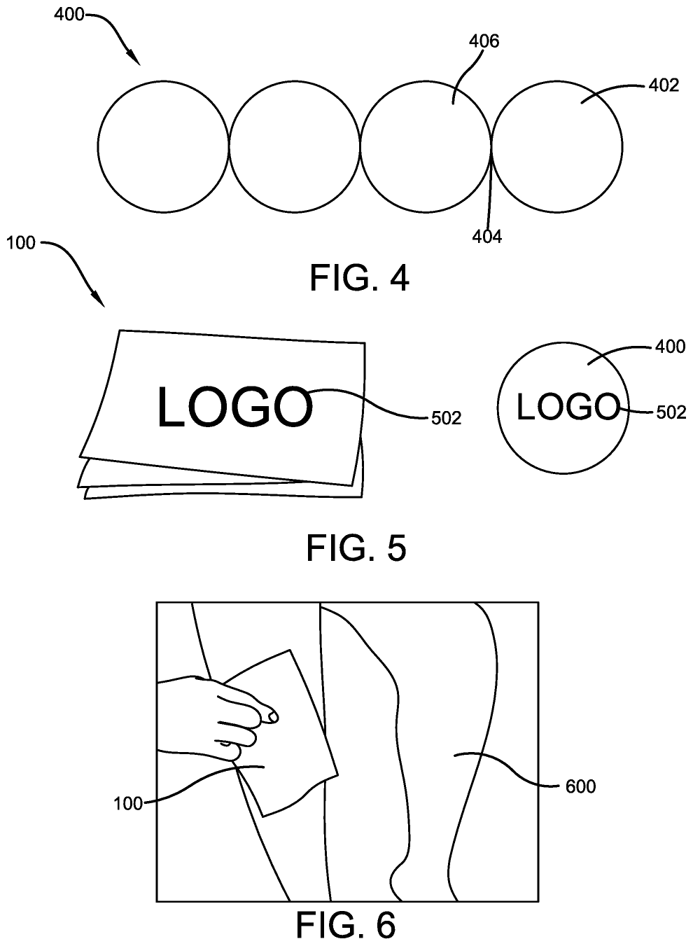

FIG. 4 illustrates a perspective view of another embodiment of an oil-infused body moisturizer sheet of the present invention in accordance with the disclosed structure. In the present embodiment, the oil-infused sheets 400 are circular in shape and each leading sheet 402 and a succeeding sheet 406 is connected by a perforated connection 404. Individual sheets can be accessed by perforating through the perforated connection 404. Each sheet is of the same approximate diameter and can be infused with a single oil or combination of two or more oils in a preconfigured composition.

FIG. 5 illustrates another embodiment of the oil-infused body moisturizer sheet of the present invention in accordance with the disclosed structure. As shown, in the present embodiment, the body sheet 100 has a Logo 502 on both surfaces or on any one surface. The logo 502 can be used for advertising and branding purpose.

FIG. 6 illustrates an oil-infused body moisturizer sheet of the present invention used for applying oil on legs of a user. In the present embodiment, the body moisturizer sheet 100 is used to apply oil on the legs and knees of a user 600. Based on the requirements of the user 600, the body moisturizer sheet 100 infused with a specific oil or a combination of specific oils can be used by the user 600.

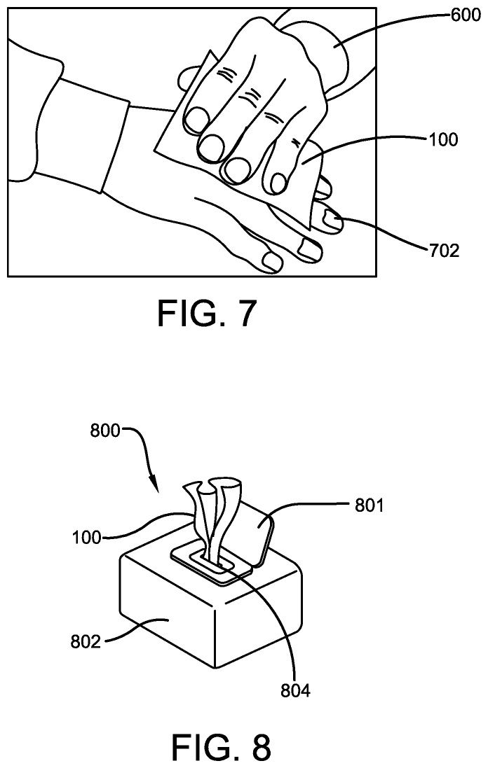

FIG. 7 illustrates an oil-infused body moisturizer sheet of the present invention used for applying oil on arms of a user. In the present embodiment, the body moisturizer sheet 100 is used to apply oil on the arms 702 of a user 600. Based on the requirements of the user 600, the body moisturizer sheet 100 infused with a specific oil or a combination of specific oils can be used by the user 600 to apply oil on the arms 702.

FIG. 8 illustrates oil-infused body moisturizer sheets of the present invention stored in a protective storage case to keep the sheets fresh, and protected from ambient air, and accessible from an opening of the protective storage case. The exemplary cuboidal-shaped container 800 stores a plurality of body moisturizer sheets 100 of the present invention. The container 800 or the dispenser 800 features a body 802 generally made up of durable and flexible plastic-based packaging material such as poly bags and the like. The packaging material for the container 800 forms a complete package for the pre-infused body moisturizer sheets 100. The container 800 features a sealable and resealable lid 801 at the top surface of the container body 802. The lid 801 is pivotally hinged on an edge. The lid 801 is an airtight seal and is at least sufficiently sealed to prevent spoilage and drying out of the moisturizer sheets 100 stored inside the container 800. The moisturizer sheets 100 are generally stacked interfolded or integrally interconnected within the container 800.

Further, the container or dispenser 800 may be of any suitable shape and size, including but not limited to, rectangular, cubic, pyramidal, cylindrical, spherical or any other shape that allows a stack or roll of body sheets 100 to be stored within. The container 800 is portable, lightweight, and easily carried when leaving home. Suitable materials may include flexible materials. The container 800 may be of any color and design as per the desires and needs of the user and manufacturers.

To use body moisturizer sheets 100 of the present invention, a user selects a sheet of a specific oil or a combination of oils, then opens a sealed lid 801 of the container or dispenser 800, and pulls out an individual sheet 100 through an opening 804 of the container. Next, the user holds the wipe with one or both hands and applies the sheet 100 over the desired body part such that the oil-infused in the sheet 100 is transferred to the desired body part. Once the sheet 100 is used, the user may dispose of the used sheet by throwing it in a dustbin or flushing it down a toilet. It is to be appreciated that an infused sheet 100 is more effective in applying oils than trying to pour some oil onto a cloth and then wiping the intended body part.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “oil infused body moisturizer sheet”, “body moisturizer sheet”, “pre-infused body moisturizer sheet” and “body sheet” are interchangeable and refer to the oil infused body moisturizer sheet 100, 400 of the present invention.

Notwithstanding the forgoing, the oil-infused body moisturizer sheet 100, 400 of the present invention can be of any suitable size and configuration as is known in the art without affecting the overall concept of the invention, provided that it accomplishes the above-stated objectives. One of ordinary skill in the art will appreciate that the size, configuration and material of the oil infused body moisturizer sheet 100, 400 as shown in the FIGS. are for illustrative purposes only, and that many other sizes and shapes of the oil-infused body moisturizer sheet 100, 400 are well within the scope of the present disclosure. Although the dimensions of the oil-infused body moisturizer sheet 100, 400 are important design parameters for user convenience, the oil-infused body moisturizer sheet 100, 400 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method for forming and using a moisturizer treatment sheet, the method comprising the steps of:
mixing at least three carrier oils with at least one essential oil in a container to form a composition;
placing a sheet into said container;
soaking said sheet in said composition;
removing said sheet from said container;
allowing an excess of the composition to drain from said sheet;
packaging said sheet;
removing said sheet from said package when moisturizing is needed; and
rubbing said sheet over a desired skin body part to transfer said composition onto the desired skin body part, wherein said carrier oils are selected from the group consisting of mineral oil, almond oil, and coconut oil, and further wherein said essential oil is selected from the group consisting of castor oil, lemon oil, lavender oil, rosemary oil, and tea tree oil.

2. The method of claim 1, wherein the mineral oil comprises from about 10% to about 99% by weight of the composition, the castor oil comprises from about 10% to about 99% by weight of the composition, the almond oil comprises from about 20% to about 80% by weight of the composition; the coconut oil comprises from about 20% to about 80% by weight of the composition, and the essential oils comprise from about 2.0% to about 10% by weight of the composition.

3. The method of claim 1, wherein the mineral oil comprises from about 50% to about 70% by weight of the composition.

4. The method of claim 1, wherein the castor oil comprises from about 50% to about 70% by weight of the composition.

5. The method of claim 1, wherein the almond oil comprises from about 40% to about 60% by weight of the composition.

6. The method of claim 1, wherein the coconut oil comprises from about 40% to about 60% by weight of the composition.

7. The method of claim 1, wherein the essential oils comprise from about 2.0% to about 6% by weight of the composition.

8. The method of claim 1, wherein the essential oils comprise at least two oils selected from the group consisting of: castor oil, lemon oil, lavender oil, rosemary oil, and tea tree oil.

9. The method of claim 1, wherein the essential oils comprise at least three oils selected from the group consisting of: castor oil, lemon oil, lavender oil, rosemary oil, and tea tree oil.

10. The method of claim 1, wherein the essential oils comprise at least four oils selected from the group consisting of: castor oil, lemon oil, lavender oil, rosemary oil, and tea tree oil.

11. The method of claim 1, wherein the essential oils comprise five oils selected from the group consisting of: castor oil, lemon oil, lavender oil, rosemary oil, and tea tree oil.

12. The method of claim 1, wherein said package includes a continuous roll of said sheets connected by perforations.

13. The method of claim 1, wherein said package includes a plurality of said sheets interfolded together in an airtight container including a lid for selective removal of an individual said sheet.

14. A method of treating an individual having dry skin comprising the steps of:
infusing a sheet with a composition comprised of a carrier oil and an essential oil;
rubbing said sheet over a desired skin body part to transfer said composition onto the skin body part;
wherein said carrier oil consists of a mineral oil, an almond oil, and a coconut oil;
wherein said essential oil consists of a castor oil, a lemon oil, a lavender oil, a rosemary oil, and a tea tree oil;
wherein said mineral oil comprises from about 10% to about 99% by weight of the composition;
wherein said castor oil comprises from about 10% to about 99% by weight of the composition;
wherein said almond oil comprises from about 20% to about 80% by weight of the composition;
wherein said coconut oil comprises from about 20% to about 80% by weight of the composition;
wherein said lemon oil comprises from about 0.5% to about 10% by weight of the composition;
wherein said lavender oil comprises from about 0.5% to about 10% by weight of the composition;
wherein said rosemary oil comprises from about 0.5% to about 10% by weight of the composition; and
wherein said tea tree oil comprises from about 0.5% to about 10% by weight of the composition.

15. The method of claim 14, wherein the mineral oil comprises from about 50% to about 70% by weight of the composition, and further wherein the castor oil comprises from about 50% to about 70% by weight of the composition.

16. The method of claim 15, wherein the almond oil comprises from about 40% to about 60% by weight of the composition, and further wherein the coconut oil comprises from about 40% to about 60% by weight of the composition.

17. The method of claim 16, wherein the essential oils comprise from about 2.0% to about 6% by weight of the composition.

18. The method of claim 14 further comprising the step of packaging said sheets, wherein said package includes a continuous roll of said sheets connected by perforations.

19. The method of claim 14 further comprising the step of packaging said sheets, wherein said package includes a plurality of said sheets interfolded together in an airtight container including a lid for selective removal of an individual said sheet.

20. An moisturizer sheet comprising:
a sheet; and
a composition infused into the sheet, wherein the composition comprises a mineral oil, an almond oil, a coconut oil, a castor oil, a lemon oil, a lavender oil, a rosemary oil and a tea tree oil.
